(19) United States
(12) Patent Application Publication
(10) Pub. No.: US 2013/0084324 A1
(43) Pub. Date: Apr. 4, 2013

(54) ADHESION PREVENTION FABRIC

(71) Applicant: Michel Gensini, Flemington, NJ (US)

(72) Inventor: Michel Gensini, Flemington, NJ (US)

(21) Appl. No.: 13/249,978

(22) Filed: Sep. 30, 2011

(51) Int. Cl. A61K 9/70; A61P 7/04

(52) U.S. Cl. 424/445

(57) ABSTRACT

The present invention is directed to an adhesion prevention fabric having a first absorbable oxidized regenerated cellulose nonwoven fabric and a second absorbable oxidized regenerated cellulose woven or knitted fabric. Further disclosed is an adhesion prevention fabric that is useful for adhesion prevention in the presence of blood.

### FIELD OF THE INVENTION

The invention relates to adhesion prevention devices. In particular, the invention relates to adhesion prevention fabrics prepared from oxidized regenerated cellulose.

### BACKGROUND OF THE INVENTION

Adhesions, or scar tissue bridges, are the abnormal connection of two or more body surfaces by fibrin associated with ingrowth of fibroblasts. Although the specific pathogenesis is not fully understood, such adhesions are likely produced as a result of the manipulative and chemical trauma associated with surgery. Such adhesions constitute a major source of postoperative morbidity and mortality and result in serious complications for the patient. Such complications are location specific, but include infertility, intestinal obstruction, loss of range of motion in joints, and the like.

Potentially dangerous for patients and a nuisance for surgeons, adhesions have been the target of study for over a century. Previous attempts at prevention of adhesions can be classified as follows: (1) prevention of fibrin deposition; (2) removal of fibrin exudate; (3) inhibition of fibroblastic proliferation; and (4) separation of surfaces.

While each of these approaches have achieved a modicum of success, the use of physical barriers to limit tissue apposition during the healing period has yielded to date perhaps the most positive results. Early approaches to the use of physical barriers employed everything from fish bladder membranes to silver and gold foils. However, it was quickly realized that these attempts did not provide the prolonged effect necessary to prevent formation of adhesions. While more modern approaches have included the use of gels and liquids, the most promising approaches to date have utilized solid physical barriers.

One traditional approach to the use of a solid physical barrier includes the use of sheets of expanded polytetrafluoroethylene (“PTFE”) to achieve the desired physical separation, as described in U.S. Pat. No. 5,468,505 to Hubbel et al. While providing the desired physical separation of the tissues, the PTFE is nonabsorbable and therefore is not preferred. The high potential for infection caused by foreign materials left in the body is well known in the art.

Attempts to utilize physical barriers made from absorbable materials, such as polylactide, polyglycolide and their copolymers have achieved limited success partly because the porosity and fibrous nature of the material exacerbates the natural defense mechanism of the body to foreign materials. Adhesion barriers such as those marketed by GYNECARE, a division of Ethicon, Inc., Somerville, N.J. under the trade name INTERCEED Absorbable Adhesion Barrier have been more successful because they are composed of oxidized regenerated cellulose (ORC) which is less reactive with tissue. However even barriers composed of ORC described in U.S. Pat. No. 4,840,626 to Linsky, et al. have achieved only limited usage due in part to the fact that the material contains pores which do not close rapidly enough on hydration to prevent the penetration of fibrin from one side of the barrier to the other. It is this fibrin bridging from one tissue to another that initiates adhesion formation.

Multilayered devices utilizing oxidized polysaccharides have been described. In U.S. Pat. No. 7,749,204, Dhanaraj et al. describe a multilayer fabric having a first absorbable nonwoven fabric reinforced by a second absorbable woven or knitted fabric and a method for tissue repair and regeneration using the multilayer fabric. The first absorbable nonwoven fabric is comprised of fibers comprising aliphatic polyester polymers, copolymers, or blends thereof. Preferably, the first absorbable nonwoven fabric comprises a copolymer of glycolide and lactide, in an amount ranging from 70 to 95% by molar basis glycolide and the remainder lactide. In an alternative embodiment, the first absorbable nonwoven fabric comprises fibers comprised of aliphatic polyester polymers, copolymers, or blends in combination with oxidized polysaccharide fibers. The second absorbable woven or knitted fabric functions as the reinforcement fabric and comprises oxidized polysaccharides, in particular oxidized cellulose and the neutralized derivatives thereof. The examples in Dhanaraj et al. are directed to polymer fibers in the nonwoven layer with an oxidized cellulose woven reinforcement fabric. The uses described are for surgical applications to enhance the wound healing. This construct is useful as a device for tissue repair.

U.S. Pat. No. 6,500,777 to Wiseman et al. describes an adhesion-prevention barrier comprising an oxidized cellulose film made up of multiple layers of a cellulose fabric or like material and a cellulose film. Wiseman et al. disclose an oxidized cellulose multilayered bioresorbable film made by spreading a cellulose film onto a surface then combining at least one layer of a cellulose fabric material with the cellulose film with an adhesive material capable of oxidation to form a bioresorbable material to form a multi-layered film. Wiseman et al., however, does not teach, suggest, or disclose preventing adhesions in the presence of blood while achieving hemostasis.

While multilayered devices utilizing oxidized polysaccharides are known, the prior art fails to describe or suggest an absorbable multilayered fabric having a first absorbable oxidized regenerated cellulose nonwoven fabric and a second absorbable oxidized regenerated cellulose woven or knitted fabric, wherein both layers are 100% ORC. Thus a need remains for a bioresorbable physical barrier for the prevention of postsurgical adhesions which is: (1) less porous than conventional woven or knitted fabric materials; (2) capable of being easily and securely attached to the desired location; and (3) administered easily during a laproscopic procedure.

### SUMMARY OF THE INVENTION

We describe herein an adhesion prevention fabric comprising a first absorbable oxidized regenerated cellulose nonwoven fabric and a second absorbable oxidized regenerated cellulose woven or knitted fabric. The adhesion prevention fabric is useful for adhesion prevention in the presence of blood.

### BRIEF DESCRIPTION OF THE FIGURES

FIG. 1A picture of an adhesion prevention fabric of the invention comprising a first oxidized regenerated cellulose (ORC) nonwoven fabric and a second oxidized regenerated cellulose woven or knitted fabric

FIG. 2 A graph of the impact of the degree of oxidation (carboxyl content) of the ORC fabric on the time to dissolution

FIG. 3 A graph of the Overall Adhesion Scores by treatment group.

FIG. 4 A graph of the percentage of adhesion-free sites by treatment group.

FIG. 5 A graph of the percentage of left uterine horn vs. right uterine horn affected by adhesions.

### DETAILED DESCRIPTION OF THE INVENTION

We describe herein an adhesion prevention fabric comprising a first absorbable oxidized regenerated cellulose nonwoven fabric and a second absorbable oxidized regenerated cellulose woven or knitted fabric.

The device of the present invention is a hemostat and adhesion prevention barrier. The hemostat is made of ORC nonwoven fibers that stop the bleeding before the blood can pass through to the adhesion prevention fabric, which is made of ORC woven fibers. The adhesion prevention fabric then keeps other tissues from adhering to the wound site.

The nonwoven fabric and the woven fabric are each prepared from oxidized regenerated cellulose. Oxidized regenerated cellulose comprises oxidized polysaccharides, in particular oxidized cellulose and the neutralized derivatives thereof. For example, the cellulose may be carboxylic-oxidized or aldehyde-oxidized cellulose. Regenerated cellulose is preferred due to its higher degree of uniformity versus cellulose that has not been regenerated. Regenerated cellulose and a detailed description of how to make oxidized regenerated cellulose is set forth in U.S. Pat. Nos. 3,364,200, 5,180,398, and 4,626,253, the contents each of which is hereby incorporated by reference as if set forth in its entirety.

As used herein, the term “nonwoven fabric” includes, but is not limited to, bonded fabrics, formed fabrics, or engineered fabrics, that are manufactured by processes other than spinning, weaving or knitting. More specifically, the term “nonwoven fabric” refers to a porous, textile-like material, usually in flat sheet form, composed primarily or entirely of staple fibers assembled in a web, sheet or batt. The structure of the nonwoven fabric is based on the arrangement of, for example, staple fibers that are typically arranged more or less randomly. The tensile, stress-strain and tactile properties of the nonwoven fabric ordinarily stem from fiber to fiber friction created by entanglement and reinforcement of, for example, staple fibers, and/or from adhesive, chemical or physical bonding. Notwithstanding, the raw materials used to manufacture the nonwoven fabric may be yarns, scrims, netting, or filaments made by processes that include spinning, weaving or knitting.

Preferably, the nonwoven fabric is made by processes other than spinning, weaving or knitting. For example, the nonwoven fabric may be prepared from yarn, scrims, netting or filaments that have been made by processes that include spinning, weaving or knitting. The yarn, scrims, netting and/or filaments are crimped to enhance entanglement with each other and attachment to the second absorbable woven or knitted fabric. Such yarn, scrims, netting and/or filaments may be cut into staple that is long enough to entangle. The staple may be between about 0.1 and 3.0 inches long, preferably between about 0.75 and 2.5 inches, and most preferably between about 1.5 and 2.0 inches. The staple may be carded to create a nonwoven batt, which may be then needle punched or calendared into the first absorbable nonwoven fabric. Additionally, the staple may be kinked or piled.

Other methods known for the production of nonwoven fabrics may be utilized and include such processes as air laying, wet forming and stitch bonding. Such procedures are generally discussed in the Encyclopedia of Polymer Science and Engineering, Vol. 10, pp. 204-253 (1987) and Introduction to Nonwovens by Albin Turbank (Tappi Press, Atlanta Ga. 1999), both incorporated herein in their entirety by reference.

The thickness of the nonwoven fabric may range from about 0.25 to 6 mm. The basic weight of the nonwoven fabric ranges from about 0.01 to 0.2 g/in²; preferably from about 0.03 to 0.1 g/in²; and most preferably from about 0.04 to 0.08 g/in². The weight percent of first absorbable nonwoven fabric may range from about 5 to 90 percent, based upon the total weight of the adhesion prevention device.

Suitable absorbable oxidized regenerated cellulose nonwoven fabrics include absorbable hemostats, including but not limited to SURGICEL FIBRILLAR absorbable hemostat and SURGICEL SNOW absorbable hemostat each available from Johnson & Johnson Wound Management, a division of Ethicon, Inc., Somerville, N.J. Absorbable hemostats suitable for the first oxidized regenered cellulose layer have a degree of oxidation ranging from 18 to 21% in order to achieve hemostasis.

The second absorbable fabric utilized in the present invention may be woven or knitted, provided that the fabric possesses the physical properties necessary for use in contemplated applications. Such fabrics, for example, are described in U.S. Pat. Nos. 4,626,253, 5,002,551 and 5,007,916, the contents of which are hereby incorporated by reference herein as if set forth in its entirety. Suitable oxidized regenerated cellulose woven fabrics include absorbable adhesion barriers such as INTERCEED absorbable adhesion barrier available from GYNECARE, a division of Ethicon, Inc., Somerville, N.J.

In preferred embodiments, the second fabric is a warp knitted tricot fabric constructed of bright rayon yarn that is subsequently oxidized to include carboxyl or aldehyde moieties in amounts effective to provide the fabrics with biodegradability. The fabric is oxidized by reacting the cellulose with a solution of nitrogen dioxide in a perfluorocarbon solvent as described by F. Boardman et al. in U.S. Pat. No. 5,180,398. In one embodiment, the carboxyl content (degree of oxidation) ranges from about 9% to about 21% (mole/mole). In another embodiment, the carboxyl content (degree of oxidation) ranges from about 12% to about 18% (mole/mole). In yet another embodiment, the oxidized regenerated cellulose woven fabric carboxyl content (degree of oxidation) ranged from about 9.5% to about 10.5% (mole/mole).

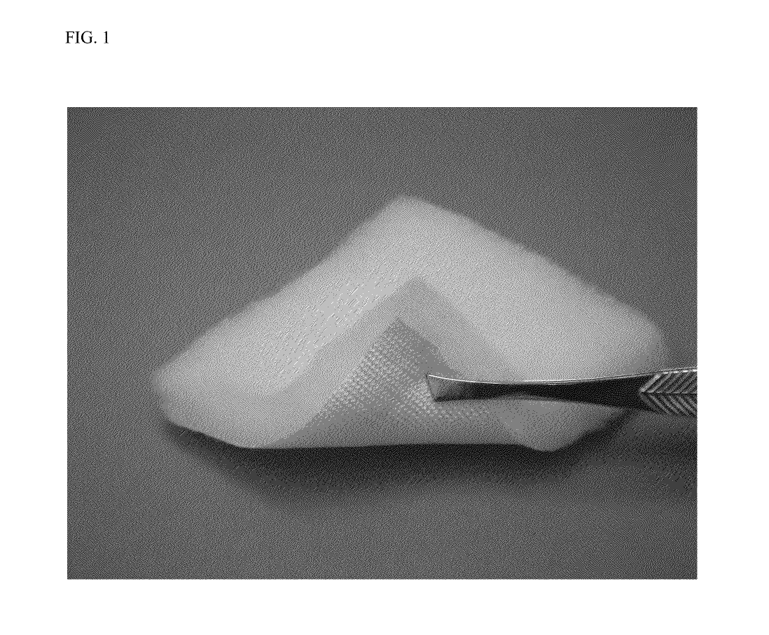

The adhesion prevention fabric is prepared by attaching the first absorbable oxidized regenerated cellulose nonwoven fabric to the second absorbable oxidized regenerated cellulose woven or knitted fabric. The first oxidized regenerated cellulose nonwoven fabric is physically attached to the second oxidized regenerated cellulose woven or knitted fabric. For example, the first nonwoven fabric may be incorporated into the second woven or knitted fabric via needle punching, calendaring, embossing or hydroentanglement. The staple of the first nonwoven fabric may be entangled with each other and imbedded in the second woven or knitted fabric. More particularly, the first nonwoven fabric may be attached to the second woven or knitted fabric such that at least about 1% of the staple of the first nonwoven fabric are exposed on the other side of the second woven or knitted fabric, preferably about 10-20% and preferably no greater than about 50%. This ensures that the first nonwoven fabric and the second woven or knitted fabric remain joined and do not delaminate under normal handling conditions. The adhesion prevention fabric is uniform such that substantially none of the second oxidized regenerated cellulose woven or knitted fabric is visibly devoid of coverage by the first oxidized regenerated cellulose nonwoven fabric (see FIG. 1).

The adhesion prevention fabric described herein is useful for preventing adhesions in the presence of blood while achieving hemostasis in part because the two oxidized regenerated cellulose layers display two different levels of oxidation. The advantages of using the adhesion prevention fabric are that the fabric may be delivered either during open surgical procedures or during laparoscopic procedures; the fabric may be applied to a specific site and does not permeate the entire surgical field; and the fabric provides adhesion prevention in the presence of blood. Specifically, the first nonwoven oxidized regenerated cellulose nonwoven fabric provides a shield for the second oxidized regenerated cellulose woven or knitted fabric. By shield or shielding we mean that the first nonwoven oxidized regenerated cellulose nonwoven fabric prevents the blood from permeating the second nonwoven oxidized regenerated cellulose nonwoven fabric. The shielding affect reduces the chance of adhesions forming as limited amount of blood is expected to reach the woven fabric and subsequently form a clot locally (for example in the case of INTERCEED, the fabric will turn black because of the presence of the clot; this clot might serves as a “bridge” for adhesions to develop).

The invention is further explained in the description that follows with reference to the drawings illustrating, by way of non-limiting examples, various embodiments of the invention.

### EXAMPLES

### Example 1

### Shielding Experiment

The shielding from blood capability of the hemostat non-woven fabric (SURGICEL FIBRILLAR) for the adhesion prevention woven fabric (INTERCEED) is evaluated in the presence of pig fresh venous blood. The test articles are one (1) square inch (1″×1″) double-layer fabrics where the non-woven fabric is needle punched into the woven one. The in vitro clotting time (Table 1.) is also measured and compared to a negative control sample (1 mL of blood deposited onto a petri dish) as well as to a positive control sample (FIBRILLAR only). Briefly, a known amount of blood ranging from 0.5 to 2.0 mL is dispensed in the center of a petri dish. The test article is then placed on top of the blood. Saline solution (ranging from 0 to 1.0 mL) is dispensed on top of the test article. Two series of experiments are run with and without any pressure applied onto the device. We observed that the clotting time for all test articles is as expected about 2 minutes. When no pressure is applied onto the test article, 1 square inch of FIBRILLAR can shield INTERCEED from up to 1.75 mL of blood. When pressure is applied onto the device, 1 square inch of FIBRILLAR can shield INTERCEED from up to 1 mL of blood. We conclude that a non woven fabric such as FIBRILLAR can decrease the exposure of an adhesion prevention woven fabric such as INTERCEED to blood.

TABLE 1

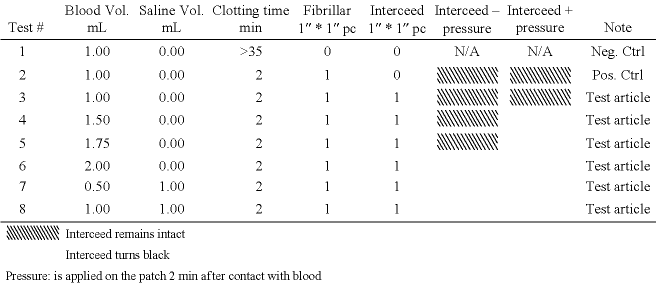

### Example 2

### Oxidized Regenerated Cellulose

Another aspect of this invention is to increase the absorption time of the ORC barrier (woven fabric) to increase its residence time in the body and therefore potentially reduce the occurrence of local adhesions at the surgical site. This can be achieved by preparing ORC compositions at different degrees of oxidation as characterized by their carboxyl content.

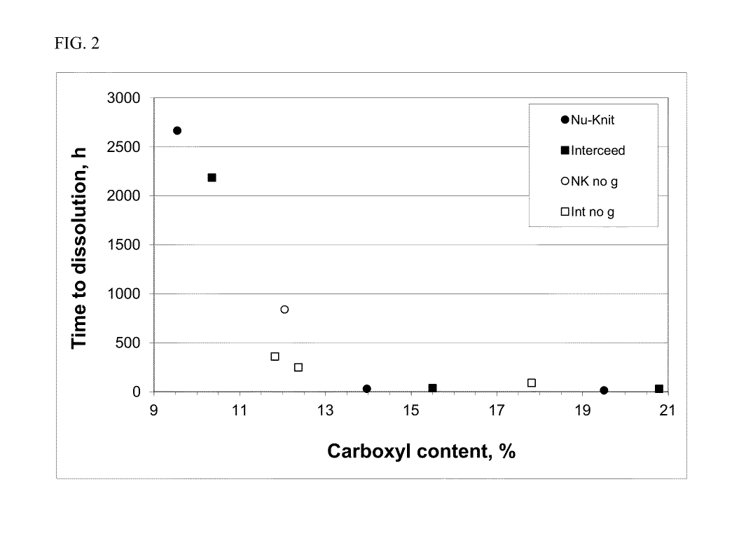

We have prepared Interceed-like and Nu-Knit-like fabrics with carboxyl contents ranging from 9% to 21% (FIG. 2). Regenerated cellulose fabric that is used to prepare INTERCEED and SURGICEL NU-KNIT (an absorbable hemostat commercially available from Johnson & Johnson Wound Management, a division of Ethicon, Inc., Somerville, N.J.) was obtained from Ethicon, Inc. The fabric was cut into samples of various sizes, typically 1″×1″. The fabric was then oxidized by reacting the cellulose with a solution of nitrogen dioxide in a perfluorocarbon solvent according to the methods described by F. Boardman et al. in U.S. Pat. No. 5,180,398. The oxidation time was varied to provide the fabrics having carboxyl content ranging from 9% to 21% (mol/mol). The carboxyl content was quantified by reverse titration with HCl in the presence of an excess of NaOH. An in vitro test was used to simulate absorption time by observing the dissolution time for the oxidized regenerated cellulose at 25° C. Time to dissolution was measured in phosphate buffered solution (pH 8) for sterilized (25 kGy gamma irradiation) and non-sterilized samples. The samples were checked visually until the samples were no longer visible. We observed that while the time to dissolution remains constant (approx. 2 days) for carboxyl contents above 14%, the time to dissolution significantly increases in a linear fashion (carboxyl content below 14%) to approximately 125 days when the carboxyl content drops off to around 9%. This can be used as a guide to select a degree of oxidation that will allow for the ORC fabric to remain at the surgical site for a time longer than the one necessary for blood clots to be absorbed.

### Example 3

### Double Uterine Horn Model for Evaluation of the Prevention of Post-Surgical Adhesions

The objective of this experiment is to evaluate the efficacy of the adhesion prevention fabric described herein in comparison to controls using a rabbit bleeding uterine horn model.

Animals:

Forty nine, female New Zealand White rabbits, 2.4-2.7 kg, were purchased from Irish Farms (Norco, Calif.) and quarantined for at least 2 days prior to use. Seven rabbits were randomized into seven treatment groups prior to initiation of surgery. The rabbits were housed on a 12:12 light:dark cycle with food and water available ad libitum.

Materials:

The treatment groups 2 to 7 are described below. The sutures used to close the muscle and skin were 3-0 polyglactin 910 suture sold under the tradename VICRYL (Ethicon, Inc., Somerville, N.J.).

Group 2 was commercially available INTERCEED Oxidized Regenerated Cellulose (ORC) for the prevention of adhesions following surgery, manufactured and distributed by Ethicon, Inc. (25×100 mm patch/horn).

Group 3 was an Interceed L which is an Interceed-like fabric oxidized at a lower level (about 10.35%) than commercially available INTERCEED. Samples prepared as described in Example 2 were provided by Ethicon, Inc. This material was sterilized by gamma irradiation (29 KGy) (25×100 mm patch/horn).

Group 4 was an Interceed L prepared as described in Example 2 and a sheet of commercially available FIBRILLAR. The sheet of FIBRILLAR was wrapped around the uterine horn then the Interceed L was wrapped over the FIBRILLAR and sutured in place with 4.0 polyglactin 910 suture sold under the tradename VICRYL (Ethicon, Inc., Somerville, N.J.) (30×100 mm patch/horn).

Group 5 was a commercially available human fibrin sealant sold under the tradename EVICEL manufactured by Omrix Biopharmaceuticals and distributed by J&J Wound Management. EVICEL was used according to the package insert instructions (1 mL/horn).

Group 6 was commercially available EVICEL as described in Group 5 was admixed with Tranilast. Tranilast is also known as N-(3,4-dimethoxycinnamoyl) anthranlic acid, which is a compound known for treating inflammation, allergies and asthma. Tranilast has been shown to be useful in reducing or preventing formation of adhesions between tissue surfaces in body cavities following surgical procedures when administered directly to the tissue and body cavity to inhibit the formation of post-operative adhesions (see US Patent Publication Number US20050106229). (25 mg+1 mL/horn).

Group 7 was an adhesion prevention barrier gel sold under the tradename INTERCOAT (Ethicon, Inc., Somerville, N.J.). INTERCOAT is a carboxymethylcellulose (97%) (Ca²⁺ crosslinked)+polyethylene oxide (3%) gel currently marketed in Europe by Ethicon, Inc. for the prevention of adhesions following surgery (2 mL/horn).

Study Design:

Bleeding Double Uterine Horn Model:

Rabbits were anesthetized with a mixture of 55 mg/kg ketamine hydrochloride and 5 mg/kg Rompum intramuscularly. Following preparation for sterile surgery, a midline laparotomy was performed. The uterine horns were exteriorized and traumatized by abrasion of the serosal surface with gauze until punctate bleeding developed. Ischemia of both uterine horns was induced by removal of the collateral blood supply. The remaining blood supply to the uterine horns was the ascending branches of the utero-vaginal arterial supply of the myometrium. At the end of surgery, no treatment, or one of three ORC patches (wrapped individually around the uterine horns and trimmed to fit when necessary), or a sealant, or a gel was administered. The horns were then returned to their normal anatomic position and the midline sutured with 3-0 polyglactin 910 suture sold under the tradename VICRYL, by Ethicon, Inc., Somerville, N.J.

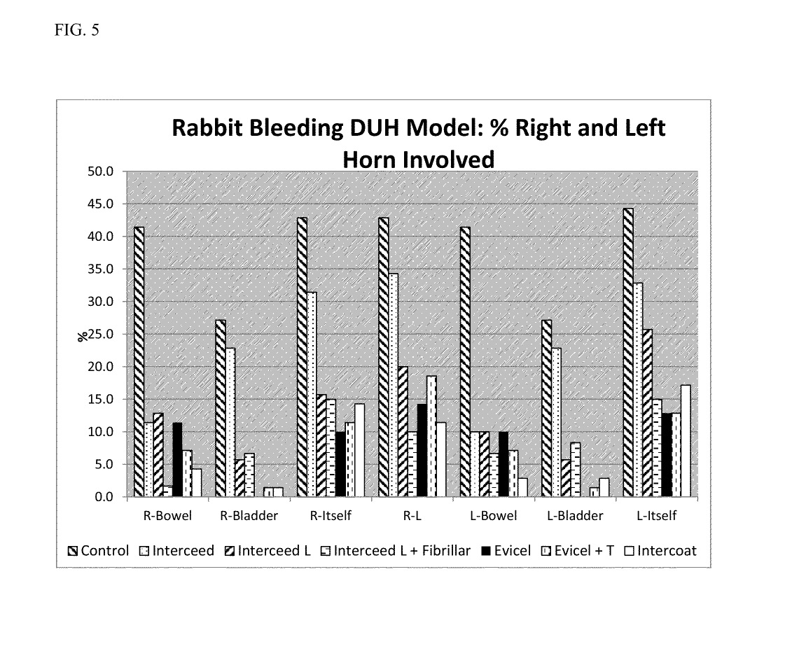

After 21 days, the rabbits were euthanized and the percentage of the area of the horns adherent to various organs determined (see Table 3, FIG. 5). In addition, the tenacity of the adhesions were given an overall score using the following system: 0=No Adhesions; 1=mild, easily dissectable adhesions; 2=moderate adhesions; non-dissectable, does not tear the organ; 3=dense adhesions; non-dissectable, tears organ when removed.

In addition an overall score which takes into account all of the above data was given to each rabbit. The scoring system described in Table 2.

TABLE 2

| Adhesion | |
|---|---|
| Score | Description |
| 0 | No adhesions |
| 0.5 | Light, filmy pelvic adhesions involving only one organ, |
| | typically only 1 or 2 small adhesions |
| 1.0 | Light, filmy adhesions, not extensive although slightly more |
| | extensive than 0.5 |
| 1.5 | Adhesions slightly tougher and more extensive than a 1 rating |
| 2.0 | Tougher adhesions, a little more extensive, uterine horns |
| | usually have adhesions to both bowel and bladder |
| 2.5 | Same as 2, except the adhesions are usually not filmy at any |
| | site and more extensive |
| 3.0 | Tougher adhesions than 2, more extensive, both horns are |
| | attached to the bowel and bladder, some movement of the |
| | uterus possible |
| 3.5 | Same as 3, but adhesions slightly more extensive and tougher |
| 4.0 | Severe adhesions, both horns attached to the bowel and |
| | bladder, unable to move the uterus without tearing the |
| | adhesions |

TABLE 3

| | % Horn Involved | | | | | | | | |
|---|---|---|---|---|---|---|---|---|---|
| | Right Horn | | | | Left Horn | | | | |
| Group | Bowel | Bladder | Itself | Left | Bowel | Bladder | Itself | Right | Overall |
| Neg. Ctrl | 41.4 ± 6.7 | 27.1 ± 7.5 | 42.9 ± 2.9 | 42.9 ± 3.6 | 41.4 ± 6.7 | 27.1 ± 7.5 | 44.3 ± 3.0 | 42.9 ± 3.6 | 44.7 ± 0.9 |
| INTERCEED | 11.4 ± 6.3 | 22.9 ± 4.7 | 31.4 ± 2.6 | 34.3 ± 3.7 | 10.0 ± 6.5 | 22.9 ± 4.7 | 32.9 ± 4.2 | 34.3 ± 3.7 | 35.8 ± 2.5 \hat{ } |
| Interceed L | 12.9 ± 3.6 | 5.7 ± 3.0 | 15.7 ± 4.8 | 20.0 ± 4.9 | 10.0 ± 3.8 | 5.7 ± 3.0 | 25.7 ± 5.3 | 20.0 ± 4.9 | 26.2 ± 3.6 \hat{ } |
| Interceed L + | 1.7 ± 1.7 | 6.7 ± 3.3 | 15.0 ± 5.0 | 10.0 ± 3.6 | 6.7 ± 4.2 | 8.3 ± 4.0 | 15.0 ± 5.0 | 10.0 ± 3.7 | 19.2 ± 3.1 \hat{ } |
| FIBRILLAR | | | | | | | | | |
| FIBRIN | 11.4 ± 4.0 | 0 ± 0 | 10.0 ± 4.4 | 14.3 ± 4.8 | 10.0 ± 4.4 | 0 ± 0 | 12.9 ± 4.2 | 14.3 ± 4.8 | 15.7 ± 3.2 |
| Sealant | | | | | | | | | |
| FIBRIN | 7.1 ± 3.6 | 1.4 ± 1.4 | 11.4 ± 4.0 | 18.6 ± 4.6 | 7.1 ± 3.6 | 1.4 ± 1.4 | 12.9 ± 6.1 | 18.6 ± 4.6 | 15.5 ± 5.6 |
| Sealant + | | | | | | | | | |
| TRANILAST | | | | | | | | | |
| INTERCOAT | 4.3 ± 2.0 | 1.4 ± 1.4 | 14.3 ± 5.3 | 11.4 ± 2.6 | 2.9 ± 1.8 | 2.9 ± 1.8 | 17.1 ± 5.2 | 11.4 ± 2.6 | 13.6 ± 2.9 |

+ This is the mean and standard error of the ranks of the overall score.
\hat{ } Brownish black material on horns

The rabbits were scored by two independent observers that were blinded to the prior treatment of the animal. If there was disagreement as to the score to be assigned to an individual animal, the higher score was given.

Statistical Analysis:

The overall scores were analyzed by rank order analysis and analysis of variance on the ranks The percentage area of the horns involved to the various organs were compared by Student's t test or one way analysis of variance.

### Results

All animals, but one, survived to necropsy and had no clinical observations. One animal that received Interceed L+FIBRILLAR died 18 days after surgery without symptoms. There was no evidence that this was treatment related.

TABLE 4

| | # Sites Free/Possible | | |
|---|---|---|---|
| Treatment Group | Sites | % Sites Free | P Value |
| Surgery Control | 2/56 | 3.6 | |
| Interceed | 11/56 | 19.6 | 0.0156 |
| Interceed L | 18/56 | 32.1 | 0.0001 |
| | | | 0.1951+ |
| Interceed L + Fibrillar | 21/48 | 43.8 | <0.0001 |
| | | | 0.0105+ |
| Fibrin Sealant | 28/56 | 50.0 | <0.0001 |
| | | | 0.0013+ |
| Fibrin Sealant + Tranilast | 25/56 | 44.6 | <0.0001 |
| | | | 0.0081+ |
| Intercoat | 27/56 | 48.2 | <0.0001 |
| | | | 0.0025+ |

+Analysis compared with Interceed.

### CONCLUSIONS

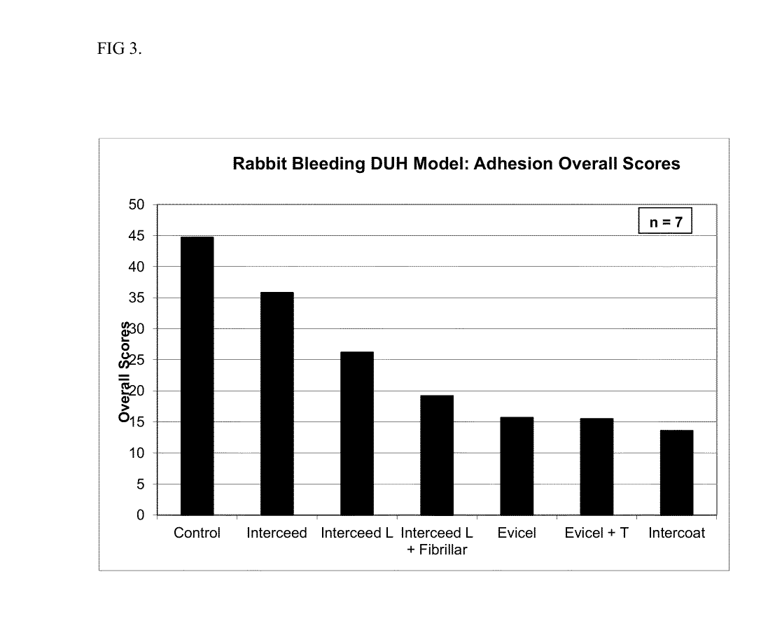

All treatment groups tested reduced the adhesion score compared to control (FIG. 3). All treatment groups with the exception of Interceed L reduced the incidence of adhesion formation and reduced the overall adhesion score when compared with INTERCEED, the current standard of care. All treatment groups with the exception of INTERCEED, reduced the overall score of adhesion formation compared with control.

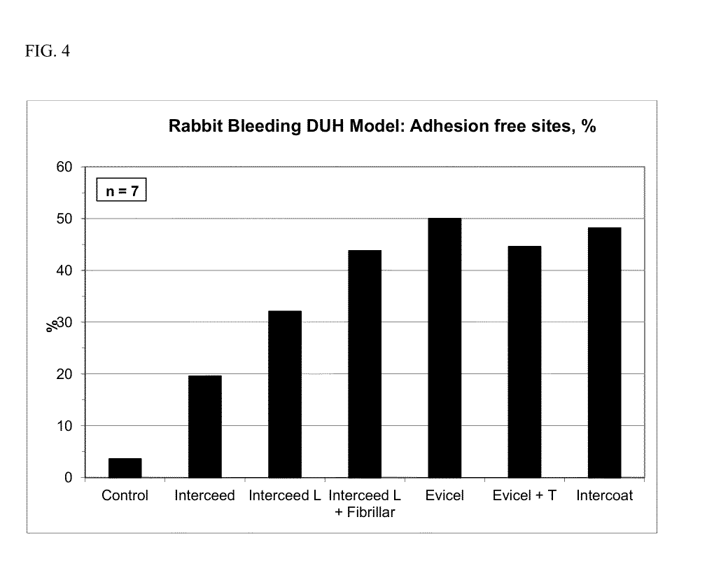

Regarding adhesion free sites (see Table 4, FIG. 4), using ANOVA followed by Tukey's multiple comparisons analysis, all treatments with the exception of INTERCEED and Interceed L were significantly different from control and there was no difference between the remaining therapies on either of these parameters. The Interceed L plus FIBRILLAR device performed similarly to the fibrin sealant and fibrin sealant plus Tranilast. Using a device approach has obvious advantages over a biologic or drug treatment including, simplified regulatory pathway and cost effectiveness.

Minimal brownish-black material was present in animals treated with a form of oxidized regenerated cellulose. While this was most extensive in the animals treated with Interceed L plus FIBRILLAR, it did not negatively impact the adhesion score or percentage of adhesion free sites. This result further demonstrates that the FIBRILLAR layer shields the Interceed L layer from the local bleeding site, reducing the incidence of adhesions and the adhesion score.

While the present invention has been described and illustrated by reference to particular embodiments and examples, those of ordinary skill in the art will appreciate that the invention lends itself to variations not necessarily illustrated herein. For this reason, then, reference should be made solely to the appended claims for purposes of determining the true scope of the invention.

We claim:

1. A device for adhesion prevention comprising a first absorbable nonwoven oxidized regenerated cellulose layer and a second absorbable woven oxidized regenerated cellulose layer attached to the first absorbable nonwoven oxidized regenerated cellulose layer.

2. The device of claim 1 wherein the first nonwoven oxidized regenerated cellulose layer is an absorbable hemostat.

3. The device of claim 1 wherein the second woven oxidized regenerated cellulose layer is an absorbable adhesion prevention barrier.

4. The device of claim 1 wherein the first nonwoven oxidized regenerated cellulose layer is an absorbable hemostat and the second woven oxidized regenerated cellulose layer is an absorbable adhesion prevention barrier.

5. The device of claim 2 wherein the absorbable hemostat has a carboxyl content of 18 to 21%.

6. The device of claim 3 wherein the absorbable adhesion prevention barrier has a carboxyl content of 9% to 21%.

7. The device of claim 4 wherein the absorbable hemostat has a carboxyl content of 18 to 21% and the absorbable adhesion prevention barrier has a carboxyl content of 9% to 21%.

8. The device of claim 1 wherein the first absorbable nonwoven oxidized regenerated cellulose layer has a thickness of about 0.25 to 6 mm.

9. The device of claim 1 wherein the first absorbable nonwoven oxidized regenerated cellulose layer has a basic weight of about 0.01 to 0.2 g/in².

10. The device of claim 1 wherein the first absorbable nonwoven oxidized regenerated cellulose layer has a weight percent from about 5 to 90 percent of a total weight of the device.

11. A method of making a device for adhesion prevention comprising the steps of:
providing a regenerated cellulose woven fabric;
oxidizing the regenerated cellulose woven fabric with a solution of nitrogen dioxide in a perfluorocarbon solvent to achieve an oxidized regenerated cellulose woven fabric having a carboxyl content ranging from 9% to 21%;
providing an oxidized regenerated cellulose nonwoven fabric; and
attaching the oxidized regenerated cellulose nonwoven fabric to the oxidized regenerated cellulose woven fabric.

12. The method of claim 11 wherein the oxidized regenerated cellulose woven fabric is an absorbable adhesion prevention barrier.

13. The method of claim 11 wherein the oxidized regenerated cellulose nonwoven fabric is an absorbable hemostat.

14. The method of claim 11 wherein the oxidized regenerated cellulose nonwoven fabric is an absorbable hemostat and the oxidized regenerated cellulose woven fabric is an absorbable adhesion prevention barrier.
